(19) United States
(12) Patent Application Publication
(10) Pub. No.: US 2005/0163732 A1
(43) Pub. Date: Jul. 28, 2005

(54) Nail makeup or nail care kit

(76) Inventors: Ronald Ramin, Paris (FR); Roberto Cavazzuti, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/853,001

(22) Filed: May 25, 2004

Related U.S. Application Data
(60) Provisional application No. 60/474,244, filed on May 30, 2003.

(30) Foreign Application Priority Data
May 26, 2003 (FR) 03 06357

(51) Int. Cl. A61K 7/04

(52) U.S. Cl. 424/61

(57) ABSTRACT

A nail makeup or nail care kit comprising: i) at least one first composition comprising, in at least one first cosmetically acceptable medium, at least one first film-forming polymer, wherein the at least one first composition is capable of forming a film having a glass transition temperature Tg ranging from 25 to 60° C. and ii) at least one second composition comprising at least one second cosmetically acceptable medium, wherein the at least one second composition has a solids content of greater than or equal to 15% by weight, relative to the total weight of the at least one second composition; and processes comprising applying the nail makeup or nail care kit to a nail.

This application claims benefit of U.S. Provisional Application No. 60/474,244, filed May 30, 2003.

Disclosed herein is a nail makeup or nail care kit comprising at least one first composition and at least one second composition, for producing a film of improved staying power on the nail. Further disclosed herein is a process comprising applying to the nail the nail makeup or nail care kit.

Nail varnish compositions that can increase the staying power of nail makeup are known from the prior art. For example, Patent Application No. EP-A-1 082 952 discloses makeup compositions, for example, for the nails, comprising glass particles coated with a metallic coat, to obtain a wear-resistant makeup result with a sparkling metallic appearance. Further, Patent No. EP 0 813 858 discloses the use of silicic acid in colloidal form in a nail composition to improve the staying power and strength of the nail varnish film. Finally, Document No. WO-A-00/27347 discloses nail compositions comprising microspheres for improving the staying power and adhesion of the film of a composition deposited on the nails.

The present inventors have discovered that it may be possible to obtain nail varnish films whose overall staying power, with regard to wear resistance, friction resistance, and impact strength may be better than those obtained in the prior art using a makeup kit comprising, for example, two compositions.

Thus, disclosed herein is a nail makeup or nail care kit comprising:
i) at least one first composition comprising, in at least one first cosmetically acceptable medium, at least one first film-forming polymer, wherein the at least one first composition is capable of forming a film having a glass transition temperature Tg ranging from 25 to 60° C. and
ii) at least one second composition comprising at least one second cosmetically acceptable medium, wherein the at least one second composition has a solids content of greater than or equal to 15% by weight, relative to the total weight of the at least one second composition.

Further disclosed herein is a non-therapeutic cosmetic nail makeup or nail care process, comprising applying to the nails at least one first coat of at least one first composition comprising, in at least one first cosmetically acceptable medium, at least one first film-forming polymer, wherein the at least one first composition is capable of forming a film having a glass transition temperature Tg ranging from 25 to 60° C. and before drying the at least one first coat, applying over all or part of the at least one first coat, at least one second coat of at least one second composition comprising at least one second cosmetically acceptable medium, wherein the at least one second composition has a solids content of greater than or equal to 15% by weight, relative to the total weight of the at least one second composition.

Even further disclosed herein is a non-therapeutic cosmetic nail makeup or nail care process comprising applying to the nails at least one first coat of at least one first composition comprising, in at least one first cosmetically acceptable medium, at least one first film-forming polymer, wherein the at least one first composition is capable of forming a film having a glass transition temperature Tg ranging from 25 to 60° C.; totally or partially drying the at least one first coat; and applying, over all or part of the at least one first coat, at least one second coat of at least one second composition comprising at least one second cosmetically acceptable medium, wherein the at least one second composition has a solids content of greater than or equal to 15% by weight, relative to the total weight of the at least one second composition.

As used herein, the expression “partial drying of the at least one first coat” means a superficial but nevertheless sufficient drying of the at least one first coat in order for the at least one second coat to be able to be applied without damaging the at least one first coat.

Further disclosed herein is the use of a kit as described herein to obtain a film deposited on the nail that has at least one property chosen from improved staying power and wear resistance properties.

As used herein, the term “cosmetically acceptable medium” means a medium that is compatible with the application of makeup to the nails.

First Composition

The at least one first composition is capable of forming a film having a glass transition temperature Tg ranging from 25 to 60° C., for example, from 30 to 45° C., and thus forms on the nail a “soft” film, which may adhere well to the nail and may have good impact strength due to its ability to absorb impacts. If the Tg of the film of the at least one first composition is less than 25° C., the film is too soft and tacky and does not allow a homogeneous application of the at least one second composition on the nails. If the Tg of the film of the at least one first composition is greater than 60° C., the film may be too hard and thus brittle and may not allow a final result that is satisfying, for example, in terms of staying power and/or wear resistance properties.

Method for Measuring the Glass Transition Temperature Tg

The glass transition temperature Tg of the film of the at least one first composition is measured by DMTA (Dynamic and Mechanical Temperature Analysis) using a DMTA machine from Polymer TA Instruments (model DMA2980) on a sample of film of the at least one first composition.

The sample is prepared by pouring the at least one first-composition into a Teflon-coated mold and then drying it on a plate thermostatically maintained at 30° C. for 24 hours, under ambient humidity conditions (typically 50% ±15% RH). A film is thus obtained, from which specimens are cut out (for example, using a punch). These specimens typically have a thickness of 200 μm, a width of 10 mm and a working length of 7 mm, after drying for 24 hours.

The sample is subjected to tensile stress and to small bending stresses at a frequency of 1 Hz (for example, it is subjected to a sinusoidal displacement ±8 μm) during a temperature scan at 3° C./minute. This tensile stress is performed on the sample at temperatures ranging, for example, from −10° C. to +80° C.

The complex modulus E*=E′+iE″ of the test polymer film is determined as a function of the temperature, wherein E′ is the storage modulus and E″ is the “loss” modulus.

From these measurements, the damping power: tgδ=E″/E′, is also deduced.

Next, the curve of the tgδ values are plotted as a function of the temperature. The glass transition temperature Tg of the polymer film corresponds to the temperature for which a maximum tgδ value is obtained (corresponding to a peak).

When the curve shows at least two peaks (in this case, the polymer film has at least two relaxations), the Tg value of the test polymer film is taken as the temperature for which the curve shows the peak of highest amplitude (i.e., corresponding to the largest value of tgδ; in this case, only the “majority” Tg is considered as the Tg value of the test polymer film).

This soft film of the at least one first composition may also be characterized by its Persoz hardness. The hardness of the film is measured on a coat 300 μm thick (before drying), deposited on a glass plate. The glass plate is dried on a support and maintained at 30° C. for 23 hours in an ambient atmosphere, and then for 1 hour at 70% relative humidity. The hardness of the film obtained is measured according to ASTM standard D-43-66, or standard NF-T 30-016 (December 1991), using a Persoz pendulum. In one embodiment, the at least one first composition is capable of forming a film having a hardness of 30 to 60 seconds.

For example, in some embodiments, the at least one first composition may be capable of forming a film characterized by particular viscoelastic behaviour.

In general, a material is said to be viscoelastic when, under the effect of shear, it has both the characteristics of a purely elastic material, i.e., it is capable of storing energy, and the characteristics of a purely viscous material, i.e., it is capable of dissipating energy, and which has a time-dependent response to stresses (non-instantaneous response).

In one embodiment, the at least one first composition may be characterized by its damping power tgδ, which represents the ratio between the energy dissipated and the energy transmitted within the material.

For example, in one embodiment, the at least one first composition is capable of forming a film having a tangent delta (tgδ) damping power of greater than or equal to 0.7 at 25° C. and 20 Hz.

Methods for Measuring the Damping Power tgδ

The damping power tgδ is measured by DMTA according to the method described above, but with the following differences:
the measurements are taken on a sample of polymer film with a thickness of less than 200±50 μm, a width of about 10 mm and a length of about 7 mm, at a constant temperature of 25° C.;
the sample is subjected to tensile stress and small bending stresses (for example, it is subjected to a sinusoidal displacement of ±8 μm) during a frequency scan, the frequency ranging from 0.1 to 20 Hz. The process is thus performed in the linear domain, with low levels of bending.

These measurements allow the complex modulus E*=E′+iE″ of the film of test composition to be determined, E′ being the storage modulus and E″ the “loss” modulus.

From these measurements, the damping power: tgδ=E″/E′, is deduced.

According to one embodiment, the at least one first composition further comprises at least one solid spherical particle.

As used herein, the term “spherical particle” means a particle that is spherical or substantially spherical, for example, of ovoid shape.

One embodiment disclosed herein relates to, therefore, a nail makeup or nail care kit comprising:
i) at least one first composition comprising, in at least one first cosmetically acceptable medium, at least one first film-forming polymer and at least one solid spherical particle and
ii) at least one second composition comprising at least one second cosmetically acceptable medium, wherein the at least one second composition has a solids content of greater than or equal to 15% by weight, relative to the total weight of the at least one second composition.

Yet another embodiment disclosed herein relates to a non-therapeutic cosmetic nail makeup or nail care process comprising applying to the nails at least one first coat of at least one first composition comprising, in at least one first cosmetically acceptable medium, at least one first film-forming polymer and at least one solid spherical particle; before drying the at least one first coat, applying over all or part of the at least one first coat, at least one second coat of at least one second composition comprising at least one second cosmetically acceptable medium, wherein the at least one second composition has a solids content of greater than or equal to 15% by weight, relative to the total weight of the at least one second composition.

Another embodiment disclosed herein relates to a non-therapeutic cosmetic nail makeup or nail care process comprising applying to the nails at least one first coat of at least one first composition comprising, in at least one first cosmetically acceptable medium, at least one first film-forming polymer and at least one solid spherical particle; totally or partial drying the at least one first coat; and applying, over all or part of the at least one first coat, at least one second coat of at least one second composition comprising at least one second cosmetically acceptable medium, wherein the at least one second composition has a solids content of greater than or equal to 15% by weight, relative to the total weight of the a least one second composition.

The at least one spherical particle used in the at least one first composition may have a mean size of less than or equal to 50 μm, for example, ranging from 0.5 to 30 μm, further, for example, from 5 to 15 μm and, even further, for example, may have a mean particle size equal to 10 μm.

As used herein, the term “mean size” means the size given by the statistical particle size distribution to half the population, which is referred to as the D₅₀. Such a mean particle size can be readily determined by one of ordinary skill in the art using known techniques.

The at least one solid spherical particle may be made of at least one material chosen from glass; ceramics; aluminas; silicas; silicates, for example, aluminosilicates and borosilicates; mica; synthetic mica, such as fluorophlogopite, tungsten carbide, cellulose and derivatives thereof; silicone resins; polymers, such as polyurethanes, polyamides, acrylic polymers, polytetrafluoroethylene, and polypropylene; natural and synthetic waxes; metals, for example, steel, copper, gold, silver, and chromium; metal oxides, for example, titanium dioxide, iron oxide, zirconium oxide, cerium oxide, and alumina oxides; alloys, such as steel and brass; materials of plant origin, for example, wood; materials of mineral origin, such as quartz, lava, and alluvia; and materials derived from animal secretion, for example, coral and mother-of-pearl.

In one embodiment, the at least one solid spherical particle may be chosen from soft and hard, deformable and undeformable solid particles. In another embodiment, the at least one solid spherical particle may be chosen from solid and hollow and colorless and colored solid particles.

The hollow particles may comprise at least one material chosen from solid and non-solid materials.

The at least one solid spherical particle may be chosen from particles totally and partially coated or covered with at least one coat of material such as those described herein or of other nature.

For example, the at least one solid spherical particle may comprise at least one material chosen from materials of different and non-homogeneous nature.

Examples of the at least one solid spherical particle that may be used in the kit disclosed herein include:
hollow cellulose beads such as those sold under the name COVABEAD CLO by the company Daito;
the glass microspheres sold under the name PRIZMALITE by the company Prizmalite;
porous silica microspheres, such as those sold under the names SA-SB and SI-SB by the company Miyoshi;
the silica microbeads sold under the name SPHERON by the company Catalyst Chemicals;
the methylsilsesquioxane resin microbeads sold under the name TOSPEARL by the company General Electric Toshiba Silicones;
the nanotitanium-coated ethylene/acrylic acid copolymer microbeads sold under the name EA-209/MT100T by the company Kobo;
the polyethylene beads coated with mineral synthetic polytetrafluoroethylene waxes sold under the names MICROSILK POLYBLEND by the company Micro Powders; and
the calcium aluminium borosilicate microspheres sold under the name LUXSIL by the company Potters Industries.

For example, the at least one solid spherical particle may be chosen from glass particles, for example, glass microbeads. As used herein, the term “glass” means any glass composition, the glass generally being, in a non-limiting manner, comprised of at least one oxide chosen from silicon oxide, titanium oxide, barium oxide, aluminium oxide, calcium oxide, magnesium oxide, boron oxide, potassium oxide, sodium oxide, and zinc oxide.

In one embodiment, the spherical glass particles used in the kit disclosed herein may comprise the following oxides, in amounts indicated below:
SiO₂ in an amount ranging from 5% to 10% by weight;
TiO₂ in an amount ranging from 25% to 35% by weight;
BaO in an amount ranging from 45% to 50% by weight;
CaO in an amount ranging from 1% to 10% by weight;
B₂O₃ in an amount ranging from 2% to 10% by weight;
Al₂O₃ in an amount ranging from 0.05% to 1% by weight;
Na₂O in an amount ranging from 0.05% to 1% by weight;
and other oxides in trace amounts.

The at least one solid spherical particle may be present in an amount ranging from 0.1% to 30% by weight, for example, from 0.5% to 10% by weight, and, even further, for example, from 1% to 5% by weight, relative to the total weight of the at least one first composition.

The at least one first composition may, for example, have a dynamic viscosity, at 25° C. and at atmospheric pressure, ranging from 0.15 to 0.5 Pa.s, measured using a LV II Brookfield viscometer.

The choice of such a viscosity may be favorable with respect to the physical organization of the at least one solid spherical particle at the moment that it is applied to the nail, and, for example, promotes a uniform and continuous distribution of the at least one solid spherical particle.

After applying the at least one first composition to the nail, a film of composition having an irregular surface is thus obtained, by virtue of the presence of the at least one solid spherical particle, this irregular surface allowing better attachment of the at least one second coat and the production of a reinforced final deposit on the nail when the at least one second composition is applied.

The at least one first cosmetically acceptable medium may be chosen from organic media comprising at least one organic solvent and from aqueous media.

In one embodiment, the at least one first cosmetically acceptable medium may be an organic medium comprising at least one organic solvent, constituting an organic phase. The at least one organic solvent may be chosen from volatile and non-volatile organic solvents at room temperature. For example, the at least one organic solvent may be chosen from the following:
ketones that are liquid at room temperature, such as methyl ethyl ketone, methyl isobutyl ketone, diisobutyl ketone, isophorone, cyclohexanone, and acetone;
alcohols that are liquid at room temperature, such as ethanol, isopropanol, butanol, diacetone alcohol, 2-butoxyethanol, and cyclohexanol;
glycols that are liquid at room temperature, such as ethylene glycol, propylene glycol, pentylene glycol, and glycerol;
propylene glycol ethers that are liquid at room temperature, such as propylene glycol monomethyl ether, propylene glycol monomethyl ether acetate, and dipropylene glycol mono-n-butyl ether;
short-chain esters (comprising from 3 to 8 carbon atoms in total), such as methyl acetate, ethyl acetate, propyl acetate, n-butyl acetate, aryl acetate, and isopentyl acetate;
alkanes that are liquid at room temperature, such as decane, heptane, octane, dodecane, cyclohexane, and isododecane; and
aldehydes that are liquid at room temperature, such as benzaldehyde and acetaldehyde.

In one embodiment, the at least one organic solvent may be chosen, for example, from short-chain esters (comprising from 3 to 8 carbon atoms in total) and alcohols that are liquid at room temperature.

In one embodiment, the at least one first composition may comprise an organic solvent medium that may also comprise water, for example, in an amount ranging from 0.1% to 10% by weight, relative to the total weight of the at least one first composition.

The at least one first composition disclosed herein may also comprise at least one aqueous medium, constituting an aqueous phase, which may form the continuous phase of the at least one first composition.

The aqueous phase may comprise essentially water; it may also comprise a mixture of water and at least one water-miscible organic solvent, i.e., with a water miscibility of greater than 50% by weight at 25° C., for example, chosen from lower monoalcohols comprising from 1 to 5 carbon atoms, such as ethanol and isopropanol; glycols comprising from 2 to 8 carbon atoms, such as propylene glycol, ethylene glycol, 1,3-butylene glycol, and dipropylene glycol; C₃-C₄ ketones; and C₂-C₄ aldehydes.

The at least one first cosmetically acceptable medium may be present in an amount ranging from 1% to 90% by weight, for example, ranging from 5% to 60% by weight, and, further, for example, ranging from 15% to 40% by weight, relative to the total weight of the at least one first composition.

The at least one first composition also comprises at least one film-forming polymer, referred to as the “at least one first film-forming polymer”.

As used herein, the term “film-forming polymer” means a polymer capable, by itself or in the presence of a possible plasticizer, of forming an isolable film. Such a film-forming polymer may be dissolved or dispersed in the form of particles in the cosmetically acceptable medium of the at least one first and/or the at least one second composition.

The at least one first film-forming polymer may be chosen from free-radical polymers, polycondensates, and polymers of natural origin.

Further, for example, the at least one first film-forming polymer may be chosen from vinyl and acrylic polymers; polyurethanes; polyesters; alkyd resins; epoxyester resins; cellulose-based polymers, such as nitrocellulose, cellulose esters, such as cellulose acetate, cellulose acetopropionate, and cellulose acetobutyrate; and resins resulting from the condensation of formaldehyde with an arylsulphonamide.

When the at least one first composition comprises an organic solvent medium, the at least one first film-forming polymer may be chosen, for example, from alkyd, acrylic, and vinyl resins; polyurethanes and polyesters; celluloses and cellulose derivatives, such as nitrocellulose, cellulose esters, such as cellulose acetate, cellulose acetopropionate, and cellulose acetobutyrate; and resins resulting from the condensation of formaldehyde with an arylsulphonamide.

When the at least one first composition comprises an aqueous medium, then the at least one first film-forming polymer may be present in the form of particles dispersed in the aqueous medium, thus forming a latex or a pseudolatex.

Examples of the at least one first film-forming polymer that may be used in the aqueous medium include polyurethanes, for example, anionic polyurethanes, polyester-polyurethanes, polyether-polyurethanes, free-radical polymers, such as acrylic, styrene-acrylic and vinyl polymers, and polyesters.

The dispersion may also comprise at least one associative polymer chosen from polyurethanes and natural gums, such as xanthan gum.

Examples of polymers in aqueous dispersions include the acrylic polymer dispersions sold under the names NEOCRYL XK-90, NEOCRYL A-1070, NEOCRYL A-1090, NEOCRYL BT-62, NEOCRYL A-1079, and NEOCRYL A-523 by the company Zeneca and Dow Latex 432 by the company Dow Chemical. Aqueous polyurethane dispersions may also be used, examples of such dispersions include the polyester-polyurethanes sold under the names AVALURE UR-405, AVALURE UR-410, AVALURE UR-425, and SANCURE 2060 by the company Goodrich and the polyether-polyurethanes sold under the names SANCURE 878 by the company Goodrich and NEOREZ R-970 by the company Avecia.

The at least one first film-forming polymer may be present in a solids amount ranging from 1% to 70% by weight and, further, for example, from 10% to 45% by weight, relative to the total weight of the at least one first composition.

To improve the film-forming properties of the at least one first composition, at least one auxiliary film-forming agent may be used.

The at least one auxiliary film-forming agent may be chosen from any compounds known to those skilled in the art as being capable of satisfying the desired function, and may be chosen, for example, from plasticizers.

In addition, when the at least one first composition disclosed herein comprises at least one first film-forming polymer in the form of particles dispersed in an aqueous medium, the at least one auxiliary film-forming agent may also be chosen from coalescers.

Second Composition

The at least one second composition of the kit disclosed herein comprises at least one second cosmetically acceptable medium having a solids content of greater than or equal to 15% by weight, for example, greater than or equal to 20% by weight, and, further, for example, greater than or equal to 25% by weight, relative to the total weight of the at least one second composition.

The solids content, i.e., the content of non-volatile matter, may be measured in different ways, examples include oven-drying methods, drying methods by exposure to infrared radiation, and also chemical methods by Karl Fischer titration of the water (this last method being applicable only in the case where the volatile part of the composition comprises only water).

For example, the amount of dry matter, commonly referred to as the “solids content”, of the at least one first composition and the at least one second composition are measured by heating the sample with infrared rays having a wavelength ranging from 2 μm to 3.5 μm. The substances comprised in the compositions that have a high vapor pressure evaporate under the effect of this radiation. By measuring the weight loss of the sample, the “solids content” of the compositions can be determined. These measurements are performed using a commercial infrared desiccator LP16 from Mettler. This technique-is fully described in the documentation for the machine supplied by Mettler.

The measuring protocol is as follows:

About 1 g of the composition is spread onto a metal crucible. After placing this crucible in the desiccator, it is subjected to a nominal temperature of 120° C. for one hour. The wet mass of the sample, corresponding to the initial mass, and the dry mass of the sample, corresponding to the mass after exposure to the radiation, are measured using a precision balance.

The solids content is calculated in the following manner:
solids content=100×(dry mass/wet mass)

For example, the at least one second composition has a dynamic viscosity, at 25° C. and at atmospheric pressure, ranging from 0.15 to 1.5 Pa.s, measured using an LV II Brookfield viscometer. For example, the at least one second composition should be fluid enough to allow a second uniform coat to be obtained on the nail, and dense enough to cover the rough areas of the first coat and to allow the formation of a smooth film.

The at least one second composition, which is intended to be applied over the at least one first coat of the at least one first composition, may produce a film of varnish that has good wear resistance, for example, resistance to friction.

The resistance of the film that may be obtained with the at least one second composition of the kit disclosed herein may be measured, for example, according to AFNOR standard NF T30-015, the principle of which is recalled hereinbelow.

The test composition is applied in the form of a coat 600 μm thick (before drying) to a disc, and is then left to dry for one hour at 30° C. The film of varnish deposited on the disc is then placed in contact for one hour with abrasive discs (Taber abrasimeter), the disc having a spin speed of one revolution per second. After one hour, the disc is weighed and the loss of mass LM of product, expressed as the percentage of the weight lost relative to the initial weight, is calculated.

Consequently, in this test, the smaller the weight loss, the smaller the percentage of weight lost, and the higher the wear resistance of the composition.

The film obtained with the at least one second composition disclosed herein has a wear resistance, expressed as a loss of mass measured according to AFNOR standard NF T30-015, of less than 5% by weight.

The at least one second composition comprises at least one second cosmetically acceptable medium. The at least one second cosmetically acceptable medium may be chosen from organic media and aqueous media. The organic and aqueous media may be chosen from the organic and aqueous solvent media described above and may be present in the at least one second composition in the amounts indicated above for the at least one first composition.

Needless to say, a person skilled in the art will take care to select the at least one second cosmetically acceptable medium such that the application of the at least one second composition to the film capable of being generated by applying the at least one first composition does not affect the quality of the film.

The at least one second composition of the kit disclosed herein may further comprise at least one film-forming polymer, referred to as the “second film-forming polymer,” chosen from the film-forming polymers described above for the at least one first film-forming polymer, in a solids amount ranging from 1% to 70% by weight, relative to the total weight of the at least one second composition.

For example, the at least one second composition comprises at least one second film-forming polymer in a solids amount of greater than or equal to 5% by weight, for example, greater than 10% by weight, further, for example, greater than 15% by weight, and, even further, for example, greater than 20% by weight, relative to the total weight of the at least one second composition.

The at least one second composition of the kit disclosed herein may also comprise at least one auxiliary film-forming agent, such as those described herein for the at least one first composition.

According to one embodiment, the at least one second composition is capable of forming a film having a gloss of greater than 70, the gloss of the film being measured using a glossmeter, in a conventional manner, via the following method.

A coat 300 μm thick of the composition whose mean gloss it is desired to evaluate is spread onto a Leneta brand contrast card of reference form 1A Penopac, using an automatic spreader. The coat covers at least the white background of the card. The 20° gloss on the white background is then measured immediately (on the wet film) using a Byk Gardner brand glossmeter of reference micro Tri-Gloss.

According to another embodiment, the at least one second composition comprises at least one “matting” filler so as to obtain a film that is more or less matte. The at least one filler may be chosen from Nylon, silk powders, precipitated silicas, talc, kaolin, and mica and silica platelets. The amount and the nature of the at least one filler may be adjusted according to the desired degree of matting effect for the film of the at least one second composition.

Additives

The at least one first composition and/or the at least one second composition may also comprise at least one plasticizer. For example, the at least one plasticizer may be chosen from common plasticizers such as:
glycols and derivatives thereof, such as diethylene glycol ethyl ether, diethylene glycol methyl ether, diethylene glycol butyl ether, diethylene glycol hexyl ether, ethylene glycol ethyl ether, ethylene glycol butyl ether, and ethylene glycol hexyl ether;
glycol esters;
propylene glycol derivatives and, for example, propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether, and propylene glycol butyl ether;
acid esters, for example, carboxylic acid esters, such as citrates, phthalates, adipates, carbonates, tartrates, phosphates and sebacates; and
oxyethylenated derivatives, such as oxyethylenated oils, for example, plant oils, such as castor oil.

The amount of the at least one plasticizer may be chosen by a person skilled in the art on the basis of his or her general knowledge, so as to obtain a composition having cosmetically acceptable properties. The at least one plasticizer may be present in an amount ranging, for example, from 0.1% to 15% by weight, and, further, for example, from 0.5% to 10% by weight, relative to the total weight of the at least one first composition or the at least one second composition.

The at least one first composition and/or the at least one second composition may also comprise at least one dyestuff, which may be chosen from pulverulent compounds and dyes that are soluble in the medium of the compositions. The at least one dyestuff may be present in an amount ranging from 0.01% to 25% by weight and, for example, from 2% to 7% by weight, relative to the total weight of the at least one first composition or the at least one second composition.

The pulverulent compounds may be chosen from at least one of the pigments, nacres, and flakes usually used in nail varnishes.

The pigments may be chosen from at least one of white, colored, mineral, and organic pigments. Examples of mineral pigments include titanium dioxide, optionally surface-treated, zirconium oxide, cerium oxide, iron oxide, chromium oxide, manganese violet, ultramarine blue, chromium hydrate, ferric blue, and metallic pigments, for example, aluminium, copper and bronze. Examples of organic pigments include carbon black, D & C pigments, and lakes based on cochineal carmine, barium, strontium, calcium,. aluminium, or guanine.

Other examples of pigments having the same effect, which may also be used in the kit disclosed herein, include at least one particle chosen from particles comprising organic, mineral, natural and synthetic substrates, for example, glass, acrylic, polyester, polyurethane, and polyethylene terephthalate resins, ceramics and aluminas, and optionally coated with metallic substances, such as aluminium, gold, copper and bronze; with metal oxides, for example, titanium dioxide, iron oxide and chromium oxide; and/or with mineral and organic pigments.

The nacreous pigments may be chosen from white nacreous pigments, such as mica coated with titanium or with bismuth oxychloride, colored nacreous pigments, such as titanium mica with iron oxides, titanium mica, for example, with ferric blue or with chromium oxide, titanium mica with an organic pigment of the abovementioned type, and also fluorophlogopite with iron oxides.

Pigments with goniochromatic properties, for example, liquid-crystal and multicoat properties, may also be used.

The dyes may, for example, be chosen from Sudan red, DC Red 17, DC Green 6, β-carotene, soybean oil, Sudan brown, DC Yellow 11, DC Violet 2, DC Orange 5 and quinoline yellow.

The at least one dyestuff may also be chosen from optical brighteners.

The at least one first composition and/or the at least one second composition may also comprise at least one other ingredient or additive commonly used in cosmetic compositions and known to those skilled in the art as being able to be incorporated into a nail varnish composition.

The at least one ingredient or additive may be chosen from fibres, coalescers, thickeners, preserving agents, fragrances, oils, waxes, surfactants, antioxidants, free-radical scavengers, spreading agents, wetting agents, dispersants, antifoams, neutralizers, stabilizers, active agents chosen from essential oils, UV-screening agents/sunscreens, moisturizers, vitamins, proteins, ceramides, plant extracts, etc.

Needless to say, a person skilled in the art will take care to select this or these optional additional compound(s) and the amounts thereof such that the properties of the at least one first composition and the at least one second composition disclosed herein are not, or are not substantially, adversely affected by the envisaged addition(s).

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific examples are reported as precisely as possible. Any numerical value, however, inherently contain certain errors necessarily resulting from the standard deviation found in their respective testing measurements. The following example is intended to illustrate the present disclosure without limiting the scope as a result.

### EXAMPLE 1

### Nail Varnish Kit

a) First Composition or “Base”:

| | |
|---|---|
| Nitrocellulose | 9.5 g |
| Tributyl citrate (plasticizer) | 8 g |
| Alkyd resin | 13 g |
| Glass microspheres (Prizmalite | 2 g |
| P2415BT) | |
| Pigments | 2.5 g |
| Thickener | 1.3 g |
| Isopropyl alcohol | 7 g |
| Ethyl acetate | 25 g |
| Butyl acetate qs | 100 g |

Procedure:

The nitrocellulose was dissolved in the solvent mixture (ethyl acetate and butyl acetate), and the plasticizer (tributyl citrate) and the alkyd resin were then added to the mixture.

The thickener and the pigments were dispersed and milled if necessary (for example in a sand mill) either into all or into a portion of the above mixture. The glass microspheres were then dispersed in the mixture, after which the entire mixture was homogenized.

b) Second Composition:

| | |
|---|---|
| Cellulose acetobutyrate | 21 g |
| Tributyl citrate (plasticizer) | 5.5 g |
| Alkyd resin | 10 g |
| Acrylic resin | 2.5 g |
| Dimethicone | 0.2 g |
| Ethyl acetate | 15 g |
| Butyl acetate qs | 100 g |

The cellulose acetobutyrate was dissolved in the solvent mixture (ethyl acetate and butyl acetate), and the plasticizer (tributyl citrate), the alkyd resin and the dimethicone were then added to the mixture.

A first coat of the first composition was applied to the nail and then, after superficial drying of this first coat, a coat of the second composition was applied over this first coat and was left to dry.

The nail varnish film thus obtained was judged to have a smooth, uniform, very glossy appearance and to have good staying power and good wear resistance.

1. A nail makeup or nail care kit comprising:
i) at least one first composition comprising, in at least one first cosmetically acceptable medium, at least one first film-forming polymer, wherein the at least one first composition is capable of forming a film having a glass transition temperature Tg ranging from 25 to 60° C. and
ii) at least one second composition comprising at least one second cosmetically acceptable medium, wherein the at least one second composition has a solids content of greater than or equal to 15% by weight, relative to the total weight of the at least one second composition.

2. The kit according to claim 1, wherein the at least one first composition is capable of forming a film having a glass transition temperature Tg ranging from 30 to 45° C.

3. A nail makeup or nail care kit comprising,
i) at least one first composition comprising, in at least one first cosmetically acceptable medium, at least one film-forming polymer and at least one solid spherical particle and
ii) at least one second composition comprising at least one second cosmetically acceptable medium, wherein the at least one second composition has a solids content of greater than or equal to 15% by weight, relative to the total weight of the at least one second composition.

4. The kit according to claim 1, wherein the at least one first composition further comprises at least one solid spherical particle.

5. The kit according to claim 4, wherein the at least one solid spherical particle has a mean size of less than or equal to 50 μm.

6. The kit according to claim 5, wherein the at least one solid spherical particle has a mean size ranging from 0.5 to 30 μm.

7. The kit according to claim 6, wherein the at least one solid spherical particle has a mean size ranging from 5 to 15 μm.

8. The kit according to claim 7, wherein the at least one solid spherical particle has a mean size equal to 10 μm.

9. The kit according to claim 3, wherein the at least one solid spherical particle is made of at least one material chosen from glass, ceramics, aluminas, silicas, silicates, mica, synthetic mica, tungsten carbide, cellulose and derivatives thereof, polymers, silicone resins, natural and synthetic waxes, metals, metal oxides, alloys, materials of plant origin and of mineral origin, and materials derived from animal secretion.

10. The kit according to claim 9, wherein the at least one solid spherical particle is chosen from glass particles.

11. The kit according to claim 3, wherein the at least one solid spherical particle is present in an amount ranging from 0.1% to 30% by weight, relative to the total weight of the at least one first composition.

12. The kit according to claim 11, wherein the at least one solid spherical particle is present in an amount ranging from 0.5% to 10% by weight, relative to the total weight of the at least one first composition.

13. The kit according to claim 12, wherein the at least one solid spherical particle is present in an amount ranging from 1% to 5% by weight, relative to the total weight of the at least one first composition.

14. The kit according to claim 1, wherein the at least one first composition is capable of forming a film having a tangent delta damping power of greater than 0.7.

15. The kit according to claim 1, wherein the at least one first composition is capable of forming a film having a Persoz hardness ranging from 30 to 60 seconds.

16. The kit according to claim 1, wherein the at least one first composition has a viscosity ranging from 0.15 to 0.5 Pa.s.

17. The kit according to claim 1, wherein the at least one first cosmetically acceptable medium is chosen from organic solvent media comprising at least one organic solvent and from aqueous media.

18. The kit according to claim 17, wherein the at least one first cosmetically acceptable medium is present in an amount ranging from 1% to 90% by weight, relative to the total weight of the at least one first composition.

19. The kit according to claim 18, wherein the at least one first cosmetically acceptable medium is present in an amount ranging from 5% to 60% by weight, relative to the total weight of the at least one first composition.

20. The kit according to claim 19, wherein the at least one first cosmetically acceptable medium is present in an amount ranging from 15% to 40% by weight, relative to the total weight of the at least one first composition.

21. The kit according to claim 1, wherein the at least one first film-forming polymer is chosen from vinyl and acrylic polymers, polyurethanes, polyesters, alkyd resins, epoxyester resins, cellulose-based polymers, and resins resulting from the condensation of formaldehyde with an arylsulphonamide.

22. The kit according to claim 21, wherein the cellulose-based polymers are chosen from nitrocellulose and cellulose esters.

23. The kit according to claim 22, wherein the cellulose esters are chosen from cellulose acetate, cellulose acetopropionate, and cellulose acetobutyrate.

24. The kit according to claim 1, wherein the at least one first film-forming polymer is present, in a solids content, an amount ranging from 1% to 70% by weight, relative to the total weight of the at least one first composition.

25. The kit according to claim 24, wherein the at least one first film-forming polymer is present, in a solids content, in an amount ranging from 10% to 45% by weight, relative to the total weight of the at least one first composition.

26. The kit according to claim 1, wherein the at least one second composition has a solids content of greater than or equal to 20% by weight, relative to the total weight of the at least one second composition:

27. The kit according to claim 26, wherein the at least one second composition has a solids content of greater than or equal to 25% by weight, relative to the total weight of the at least one second composition.

28. The kit according to claim 1, wherein the at least one second composition has a viscosity ranging from 0.15 to 1.5 Pa.s.

29. The kit according to claim 1, wherein the at least one second cosmetically acceptable medium is chosen from organic solvent media comprising at least one organic solvent and from aqueous media.

30. The kit according to claim 29, wherein the at least one second cosmetically acceptable medium is present in an amount ranging from 1% to 90% by weight, relative to the total weight of the at least one second composition.

31. The kit according to claim 30, wherein the at least one second cosmetically acceptable medium is present in an amount ranging from 5% to 60% by weight, relative to the total weight of the at least one second composition.

32. The kit according to claim 31, wherein the at least one second cosmetically acceptable medium is present in an amount ranging from 15% to 40% by weight, relative to the total weight of the at least one second composition.

33. The kit according to claim 1, wherein the at least one second composition further comprises at least one second film-forming polymer.

34. The kit according to claim 33, wherein the at least one second film-forming polymer is chosen from vinyl and acrylic polymers, polyurethanes, polyesters, alkyd resins, epoxyester resins, cellulose-based polymers, and resins resulting from the condensation of formaldehyde with an arylsulphonamide.

35. The kit according to claim 34, wherein the cellulose-based polymers are chosen from nitrocellulose and cellulose esters.

36. The kit according to claim 35, wherein the cellulose esters are chosen from cellulose acetate, cellulose acetopropionate, and cellulose acetobutyrate.

37. The kit according to claim 33, wherein the at least one second film-forming polymer is present, in a solids content, in an amount greater than or equal to 5% by weight, relative to the total weight of the at least one second composition.

38. The kit according to claim 37, wherein the at least one second film-forming polymer is present, in a solids content, in an amount greater than 10% by weight, relative to the total weight of the at least one second composition.

39. The kit according to claim 38, wherein the at least one second film-forming polymer is present, in a solids content, in an amount greater than 15% by weight, relative to the total weight of the at least one second composition.

40. The kit according to claim 39, wherein the at least one second film-forming polymer is present, in a solids content, in an amount greater than 20% by weight, relative to the total weight of the at least one second composition.

41. The kit according to claim 1, wherein the at least one first composition and/or the at least one second composition further comprises at least one plasticizer.

42. The kit according to claim 41, wherein the at least one plasticizer is present in an amount ranging from 0.1% to 15% by weight, relative to the total weight of the at least one first composition or the at least one second composition.

43. The kit according to claim 42, wherein the at least one plasticizer is present in an amount ranging from 0.5% to 10% by weight, relative to the total weight the at least one first composition or the at least one second composition.

44. The kit according to claim 1, wherein the at least one first composition and/or the at least one second composition further comprises at least one dyestuff.

45. The kit according to claim 44, wherein the at least one dyestuff is present in an amount ranging from 0.01% to 25% by weight, relative to the total weight of the at least one first composition or the at least one second composition.

46. The kit according to claim 45, wherein the at least one dyestuff is present in an amount ranging from 2% to 7% by weight, relative to the total weight of the at least one first composition or the at least one second composition.

47. The kit according to claim 1, wherein the at least one first composition and/or the at least one second composition further comprises at least one ingredient or additive chosen from fibres, coalescers, thickeners, preserving agents, fragrances, oils, waxes, surfactants, antioxidants, free-radical scavengers, spreading agents, wetting agents, dispersants, antifoams, neutralizers, stabilizers, active agents chosen from essential oils, UV-screening agents/sunscreens, moisturizers, vitamins, proteins, ceramides, and plant extracts.

48. A non-therapeutic cosmetic nail makeup or nail care process comprising,
applying to the nails at least one first coat of at least one first composition comprising, in at least one first cosmetically acceptable medium, at least one first film-forming polymer, wherein the at least one first composition is capable of forming a film having a glass transition temperature Tg ranging from 25 to 60° C. and
applying over all or part of the at least one first coat, and before drying of the at least one first coat, at least one second coat of at least one second composition comprising at least one second cosmetically acceptable medium, wherein the at least one second composition has a solids content of greater than or equal to 15% by weight, relative to the total weight of the at least one second composition.

49. A non-therapeutic cosmetic nail makeup or nail care process comprising,
applying to the nails at least one first coat of at least one first composition comprising, in at least one first cosmetically acceptable medium, at least one first film-forming polymer, wherein the at least one first composition is capable of forming a film having a glass transition temperature Tg ranging from 25 to 60° C.;
totally or partially drying the at least one first coat; and
applying, over all or part of the at least one first coat, at least one second coat of at least one second composition comprising at least one second cosmetically acceptable medium, wherein the at least one second composition has a solids content of greater than or equal to 15% by weight, relative to the total weight of the at least one second composition.

50. A non-therapeutic cosmetic nail makeup or nail care process comprising,
applying to the nails at least one first coat of at least one first composition comprising, in at least one first cosmetically acceptable medium, at least one first film-forming polymer and at least one solid spherical particle and
before drying the at least one first coat, applying over all or part of the at least one first coat, at least one second coat of at least one second composition comprising at least one second cosmetically acceptable medium, wherein the at least one second composition has a solids content of greater than or equal to 15% by weight, relative to the total weight of the at least one second composition.

51. A non-therapeutic cosmetic nail makeup or nail care process comprising,
applying to the nails at least one first coat of at least one first composition comprising, in at least one first cosmetically acceptable medium, at least one first film-forming polymer and at least one solid spherical particle;
totally or partially drying the at least one first coat; and
applying, over all or part of the at least one first coat, at least one second coat of at least one second composition comprising at least one second cosmetically acceptable medium, wherein the at least one second composition has a solids content of greater than or equal to 15% by weight, relative to the total weight of the at least one second composition.

52. A process for obtaining a film deposited on a nail that has at least one property chosen from improved staying power and wear resistance properties comprising applying to the nail, a nail makeup or nail care kit comprising:
at least one first composition comprising, in at least one first cosmetically acceptable medium, at least one first film-forming polymer, wherein the at least one first composition is capable of forming a film having a glass transition temperature Tg ranging from 25 to 60° C. and
at least one second composition comprising at least one second cosmetically acceptable medium, wherein the at least one second composition has a solids content of greater than or equal to 15% by weight, relative to the total weight of the at least one second composition.
